(19) United States
(12) Patent Application Publication
(10) Pub. No.: US 2009/0169531 A1
(43) Pub. Date: Jul. 2, 2009

(54) Use of symbiotic for the treatment of atopic dermatitis

(76) Inventors: Gérard Lacoste, Aurillac (FR); Hubert Desjonqueres, Pessat-Villeneuve (FR); Stéphane Patrier, Aurillac (FR)

Correspondence Address: COOPER & DUNHAM, LLP, 30 Rockefeller Plaza, 20th Floor, NEW YORK, NY, 10112, US

(21) Appl. No.: 11/991,046

(22) PCT Filed: Aug. 24, 2006
(86) PCT No.: PCT/FR2006/001979

§ 371 Date: Jan. 9, 2009

(30) Foreign Application Priority Data
Aug. 25, 2005 (FR) 0508751

(51) Int. Cl. A61K 35/74

(52) U.S. Cl. 424/93.45

(57) ABSTRACT

The invention relates to the use of a product comprising a living Lactobacillus rhamnosus bacterium combined with lactose, particularly with a lactose-based medium, for the production of a medicament or a food supplement in order to treat a patient suffering from atopic dermatitis, in which said bacterium can ferment the lactose. In the preferred embodiments of the invention, the bacterium used originates from strain Lactobacillus rhamnosus Lcr35 and is used as a probiotic combined with a prebiotic that is, for example, based on lactose and optional metabolites resulting from the fermentation of the bacterium on the culture medium thereof, for the treatment of atopic dermatitis in children.

Probiotics are living micro-organisms which are beneficial to the health of their host. This beneficial effect is explained in particular by an improvement in the equilibrium of the intestinal flora (1). The best known are lactobacillae and bifidobacteria which, by multiplying in the intestine, can reduce the potentially pathogenic bacterial population by simple competition.

Prebiotics are substances which cannot be digested by the organism and/or which have beneficial effects on the person consuming them by stimulating the growth or activity of certain non pathogenic bacteria which are naturally present in the colon.

The limited life and low survival rate of probiotics, however, remain major obstacles to their use in therapy. Thus, there is a need for suitable prebiotics and for optimized combinations of prebiotics and probiotics (symbiotics) to obtain a significant improvement in the efficacy of probiotics. Such a combination would clearly have certain advantages for the treatment of atopic disorders, in particular atopic dermatitis (AD).

There is currently no data regarding the use of prebiotics or symbiotics in the treatment of atopic dermatitis.

Atopic dermatitis is a dermatosis with a prevalence in children in Western states of between 10% and 25% (2, 3). Its evolution is chronic, frequently with improvement in adolescence. Because of the chronic nature of the prurigenic lesions which are unsightly and necessitate daily care, the moderate and severe forms can have a dramatic psychological effect. The basic treatment resides in the regular application of emollients associated with a hygieno-dietetic regime. Severe episodes necessitate the application of corticoids or immunosuppressors in a topical form. However, relapses are frequent, the chronic nature can deteriorate quality of life both for the patient and for his family, and the prolonged use of the treatments induces side effects.

The pathogenesis of atopic dermatitis resides in a dysregulation of cellular immunity with an imbalance in the Th1/Th2 ratio, to the advantage of a type Th2 response. The constant increase in the prevalence of atopic dermatitis in Western states is correlated to an improvement in hygiene. Children are less exposed to infectious agents during their first months and years of life and thus develop a preferential Th2 response (4-7). There is currently no treatment on the market which can correct this immune dysregulation.

Probiotics are live micro-organisms in the digestive tract which may have anti-allergic properties which encourage a type Th1 response (8, 9). However, not all probiotics have the same immunological properties. In a recent article on mouse dendritic cells, the bacterium Lactobacillus rhamnosus was shown to significantly induce the production of cytokines encouraging the Th1 response (namely interleukins IL-12, IL-6, the tumor necrosis factor α TNFα) without increasing the production of IL-10 (known for its active role in the Th2 response) (10). This strain is thus of great interest in the treatment of atopic dermatitis.

It has also been shown that L. rhamnosus GG is effective in the prevention of atopic dermatitis (11, 12) and to a lesser degree in the treatment of young children (13-15). These studies were carried out with very young children and the efficacy of the treatment could be due to an initial colonization of intestinal microflora. However, the immunomodulating effects of probiotics are also observed in children and adults, and could thus be effective in these age groups (16). Currently, just one study has evaluated the use of probiotics, for just 6 weeks, in children aged 6 months or more (17). While that study shows that probiotics may reduce intestinal inflammation consecutive to atopic dermatitis, the long-term efficacy of the use of probiotics in the treatment of atopic dermatitis was not addressed.

Thus, there is a distinct need for a symbiotic combination of a prebiotic and a probiotic which can ensure a good degree of survival of the probiotic and can treat atopic dermatitis.

The present invention shows the efficacy of symbiotic (Lactobacillus rhamnosus probiotic and prebiotics) in the prevention and treatment of atopic dermatitis, in particular in children over two years of age.

More particularly, the present invention concerns the use of the Lactobacillus rhamnosus Lcr35 strain (probiotic) associated with lactose (in particular in association with a lactose-based medium) in the treatment of atopic dermatitis. The term “treatment of atopic dermatitis” means the curative and/or preventive treatment. The invention also highlights the importance of lactose in the treatment of atopic dermatitis.

The Lactobacillus rhamnosus Lcr35 strain is derived from the species Lactobacillus rhamnosus (also known as Lactobacillus casei subsp rhamnosus), the latter being deposited with the ATCC with accession number ATCC 7469. The particular strain Lactobacillus rhamnosus Lcr35 may be obtained using the protocol defined in Example 1.

The present invention concerns the use of a product comprising a Lactobacillus rhamnosus bacterium in association with lactose in the treatment of atopic dermatitis. Preferably, the Lactobacillus rhamnosus bacterium is in a lactose-based medium. Preferably, the use is for the production of a drug, a food supplement, etc, to treat a patient afflicted with atopic dermatitis or for the prevention of such an affliction. Within the context of the present invention, treatment or prevention comprises reducing the allergic risk and improving the health of the patient.

Thus, the product of the invention is a combination of a probiotic (Lactobacillus rhamnosus bacterium) and a prebiotic (lactose, more specifically a lactose-based medium). Such a product can thus be termed symbiotic. Lactose is indeed known for its prebiotic effect; it arrives in the intestine in the untransformed form and thus constitutes food for certain bacterial populations in the intestinal flora, which justifies its designation as a prebiotic.

Because lactose is implicated in milk allergies, the use of lactose as a prebiotic by the present inventors is particularly original.

Preferably, the bacterium under consideration used in the product composition is capable of fermenting lactose.

The inventors have shown that the survival rate of the probiotic is very substantially increased when said probiotics is associated with an element necessary for its growth. Thus, one product of the invention comprises a probiotic capable of fermenting lactose, as well as the lactose which is necessary for the probiotic to survive.

In a particularly preferred aspect of the invention, said bacterium is the bacterium Lactobacillus rhamnosus Lcr35. Lactobacillus rhamnosus Lcr35 has the capacity of fermenting lactose, while Lactobacillus rhamnosus GG, for example, cannot.

A symbiotic as defined in the present invention may clearly contain at least two or more different probiotics. It may be a Lactobacillus rhamnosus bacterium with a bacterium of another genus, or it may be at least one or more bacterium from the genus Lactobacillus, of different species.

The symbiotic product as described preferably comprises a live bacterium. The term “live” means the cell integrity is conserved and that the cell processes occur or can occur if the bacterium is cultivated in a medium under suitable conditions. Any live bacterium may be seeded again onto a suitable culture medium and multiply under suitable conditions.

Thus, the present invention comprises the use of Lactobacillus rhamnosus Lcr35 in the freeze-dried or frozen form.

A product of the invention is considered to be a symbiotic. The bacterium is a probiotic, while the medium, based in particular on lactose, is a prebiotic.

The term “drug or food preparation or food supplement etc” means any pharmacologically acceptable form, in particular in the form of a preparation for a baby, a product intended for a young child, a dietetic and diet product, an oral dietetic supplement, a hygiene product, a food or dietetic substance for particular medical purposes (or special purposes), a medical device, or any other suitable form which is well known to the skilled person. The product is preferably in a galenical form which is adapted to oral administration, for example in the form of a drinkable suspension or a gelule. It may also be in the form of a powder or granules to be added to food or drink, or for sucking.

The term “atopic dermatitis” means any disorder diagnosed as being atopic dermatitis or atopic eczema and atopic disorders the symptoms of which are essentially identical and which also receive identical treatments.

The treated patient receiving the product described is preferably a human being, more preferably a child. The term “child” means any person less than 18 years of age, preferably less than 15 years of age or below 12 years of age. The patient is preferably a child more than 1 year of age, more particularly a child more than two years of age.

Alternatively, the patient under consideration may be a mammal other than human, for example a monkey, dog, cat or rabbit.

A preferred class of patients for the use of the present invention is constituted by children between 2 and 12 years of age.

Another population of patients which may be capable of receiving a product in accordance with the present invention is constituted by subjects with lactose allergies, in particular allergies to cow's milk. Preferably, the allergies under consideration are benign or moderate lactose allergies.

The term “atopic dermatitis treatment” means any action aimed at overcoming or reducing the symptoms of atopic dermatitis, and any preventative action which tends to prevent, retard or slow their appearance.

The treatment duration can be defined as a function of the evolution of the atopic dermatitis, and is preferably more than 2 weeks. It may be 3 months or more, in particular at least 6 months or one year. It may be appropriate to continue treatment for several years, for example 2, 5 or 8 years, in particular in the case of preventative application.

Preferably, in the treatment or prevention of atopic dermatitis, the product of the invention is administered at least once a week, preferably at least once a day. In the context of the present invention, a preferred dosage is three times a day.

The product of the invention may be in any suitable form. Suitable forms are well known to the skilled person. In particular, they must ensure that the probiotic survives. If the probiotic is not alive on ingestion, the beneficial effects of the symbiotic are absent. It is very important to take into account the three essential factors of atmosphere, humidity and temperature. The various modes of preserving probiotics are well known to the skilled person.

Preferably, the product is in the freeze-dried form. This form affords not only excellent preservation, but can also preserve any live bacterium forming part of the product of the invention. It is important to determine the probiotic count when obtaining a freeze-dried product of the invention.

The bacterium contained in the product may be the only freeze-dried element, but preferably, the bacterium is freeze-dried with the product containing the lactose-based medium. Clearly, the product may comprise additional constituents, in addition to the bacterium and the medium, comprising inter alia lactose. Such constituents are added before freeze-drying the other components or thereafter (case, for example, of excipients allowing the galenical form of the product to be produced).

The lactose-based medium is a medium containing at least 5% by weight of lactose, preferably at least 20%.

Said medium is preferably sterile, before seeding with the probiotic.

Preferably, said medium is a culture medium (fermentation) thus containing other substances. The medium is preferably a medium essentially constituted by milk, for example skimmed milk.

The lactose-based medium advantageously also contains potato starch. It may also contain maltose dextrin. Any other pharmacologically acceptable compound or excipient may also form part of the composition of a lactose-based medium as defined.

In a preferred embodiment of the present invention, the lactose-based medium corresponds to a culture medium compatible with the fermentation of lactose by the probiotic bacterium included in the product of the invention. Thus, in one preferred embodiment of the invention, the bacterium is cultivated on a culture medium which is optionally subsequently supplemented to form a product in accordance with the invention, i.e. a symbiotic (combination of a probiotic and a prebiotic). Such a situation is detailed in an example in the experimental section of the present application (see Example 1).

Particularly advantageously, the lactose-based medium derives from the probiotic culture medium. Thus, when producing the product, there is no point in changing the probiotic medium. This avoids too many centrifuging steps; during centrifuging, the cell walls of the micro-organisms may be damaged, which does not aid survival. By avoiding changing the probiotic medium and thus reducing the number of centrifuging steps, the probiotic stands a better chance of survival. Preferably, the probiotic used is not seeded or cultivated on MRS.

Because the probiotic corresponds to a culture medium adapted for the probiotics (bacteria), when the product of the invention is ingested (and in particular when it is in the freeze-dried form and is taken up into suspension before ingestion), the bacterium is immediately in a medium compatible with its survival. Thus, the survival rate of the probiotics is increased.

Preferably, the bacterium is cultivated for at least 4 hours, at least 12 h, preferably between 24 and 48 hours, preferably more, for example about 72 hours, on a culture medium based in particular on lactose which is then optionally supplemented to form the product of the invention.

The invention resides in the effects obtained by the symbiotic, i.e. by a combination of a probiotic (bacterium from the species Lactobacillus rhamnosus) and a prebiotic (lactose, preferably in the form of a lactose-based medium). The effects obtained are more advantageous when the bacterium has been cultured on a medium based, inter alia, on lactose. Under these conditions, the medium also contains metabolites resulting from said culture. Certain of said metabolites have particularly advantageous antimicrobial properties (31). The association of these compounds (probiotics, prebiotics and metabolites) contributes to the beneficial effect on atopic dermatitis.

In a product of the present invention, the number of bacteria or cells per gram of product is preferably in the range 1×10⁸ to 1×10¹⁰.

Before any freeze drying of the symbiotic product of the invention, it is generally in the liquid form. In such a situation, for use in the treatment of atopic dermatitis, the product preferably comprises a concentration in the medium of 1×10⁸ CFU (colony forming units or cells) per ml.

For an application in the prevention or treatment of atopic dermatitis, a patient is administered with about 1×10⁸ to 1×10¹⁰ CFU of Lactobacillus rhamnosus per dose. Several doses may be administered per day, for example in the form of 3 daily doses. The doses may be adjusted, in particular as a function of the body size of the patient to be treated and any reaction to the treatment.

As mentioned in the above paragraphs, a particularly preferred bacterium of the species Lactobacillus rhamnosus (equivalent to Lactobacillus casei subsp. rhamnosus) capable of fermenting lactose is the Lcr35 strain which may be obtained using the protocol defined in Example 1. Other protocols are known to the skilled person.

When selecting a probiotic to obtain a product of the invention, it is essential to research a bacterium which has no pathogenic effect and which is preferably known to have good properties as regards its survival rate. Another parameter to take into account is its resistance to technological processes. It may also be advantageous for the probiotic in question to be resistant to the majority of conventional antibiotics. In contrast, there will be situations when the probiotic is preferably sensitive to the majority of known antibiotics.

Further, a strain for use in the present invention must also have the capacity to colonize the intestinal flora.

The specific strain Lactobacillus rhamnosus Lcr35 is particularly preferred in the context of the present invention as it enjoys the various advantages mentioned above. It is also resistant to streptomycin, tetracyclin, erythromycin, nalidixic acid, pipemidic acid, fusidic acid, colistin, polymyxin, sulfamethoxazole and trimethoprim.

The fermentary characteristics of said strain are particularly advantageous.

Alternatively, the invention also concerns the use of lactose alone or in combination with a probiotic in the treatment or prevention of atopic dermatitis.

### KEY TO FIGURES

FIG. 1: genotyping of Lactobacillus rhamnosus Lcr35 strain;

FIG. 1A: 16S-23S rDNA intergene sequence; FIG. 1B: 16S ribosomal DNA;

FIG. 2: representation of sequenced regions of Lactobacillus rhamnosus Lcr35;

FIGS. 3A and 3B: comparison of 50 API CH metabolic profiles of Lactobacillus rhamnosus Lcr35 and Lactobacillus GG strains.

### EXAMPLE 1

### Obtaining and Packaging the Freeze-Dried Lactobacillus rhamnosus Lcr35 Strain

Reactivation:

Commercially available cells (Bacilor® from Lyocentre Laboratories) or from the Lyocentre Laboratories strain library were reactivated by seeding with 100 to 200 mg of the freeze-dried product, 10 ml of enriched (glucose 15 g/l, yeast extract 5 g/l) skimmed milk medium. This tube was placed in an oven at 37° C. for 72 hours.

1 ml of culture from the first tube was introduced into a second tube containing 10 ml of sterile enriched milk medium; the tube was placed in an oven at 37° C. for 72 hours. This operation was recommenced several times to obtain as large a population as possible, more than 1×10⁹ cells per ml.

Preparation of Stock Solution:

1 ml of this reactivated strain was introduced into a flask containing 180 ml of sterilized enriched skimmed milk then cultivated at 37° C. for 72 hours; this stock solution was used to seed the flasks.

Culture Medium:

In order to have available a freeze-dried culture of Lactobacillus casei var. rhamnosus (also known as Lactobacillus rhamnosus) in a suitable titer, it was produced simultaneously using 2 different culture processes:
on simplified medium;
on enriched medium.

a) Constituents of Media:

1. Simplified Medium:

| | | |
|---|---|---|
| powdered skimmed milk | 100 | g |
| water, qsp | 1.000 | l |

2. Enriched Medium:

| | | |
|---|---|---|
| powdered skimmed milk | 16.650 | g |
| glucose | 4.450 | g |
| manganese sulfate | 0.028 | g |
| sodium citrate | 0.560 | g |
| monopotassium phosphate | 1.780 | g |
| dipotassium phosphate | 3.560 | g |
| beer yeast autolysate | 1.670 | g |
| water, qsp | 1.000 | l |

b) Preparation and Sterilization of Media:

1. Simplified Medium:

Formula:

| | | |
|---|---|---|
| powdered skimmed milk | 100 | g |
| water, qsp | 1.000 | l |

Method:

200 liters of water is measured into a container and 28 kg of powdered skimmed milk is added in small fractions, with gentle stirring. After complete dispersion, it is adjusted to 280 l with water. This milk is dispensed into containers which are correctly sealed then sterilized in an autoclave for 30 minutes at 115° C.

2. Enriched Medium:

Formula for a 2000 Liter container

| | | |
|---|---|---|
| powdered skimmed milk | 30.00 | kg |
| glucose | 8.00 | kg |
| manganese sulfate | 0.05 | kg |
| sodium citrate | 1.00 | kg |
| monopotassium phosphate | 3.20 | kg |
| dipotassium phosphate | 6.40 | kg |
| beer yeast autolysate | 3.00 | kg |
| water, qsp | 1800.00 | l |

Method:

Place 1000 l of water in a container then add the milk, stirring rapidly; after complete dispersion, add the glucose, manganese sulfate, sodium citrate, mono- and di-potassium phosphates and finally the beer yeast autolysate. After complete dispersion, adjust to 1800 liters with water.

Finally, still with stirring, sterilize the medium at 115° C. for 30 minutes then cool to 37° C.

Seeding and Culture

a) Simplified Medium:

Introduce 1 to 2 ml of pre-culture from the stock solution per container, in a hood with a vertical laminar air flow.

Next, these containers are placed in bacteriological ovens at 37° C. plus or minus 2° C. for 72 hours.

b) Enriched Medium:

2.4 liters of pre-culture from the stock solution is introduced into each 1800 liter container. Maintain the temperature at 37° C. using a suitable thermostatted device for 48 hours with gentle stirring.

Preparation of Culture for Freeze Drying:

a) Simplified Medium:

Constituents:

| | |
|---|---|
| culture of Lactobacillus casei var. rhamnosus | 136.8 l |
| potato starch | 35.0 kg |
| lactose | 15.0 kg |
| suspension of calcium hydroxide, qs for neutralization | pH = 7.0 |

Preparation Method:

The culture obtained is poured into a 200 liter tank under a laminar flow hood, with vigorous stirring to break the curd, and then add the potato starch and lactose with vigorous stirring. When completely dispersed, measure the pH which should be close to 3. Next, neutralize the mixture to a pH of 7.0±0.1 using calcium hydroxide.

b) Enriched Medium

Constituents:

| | | |
|---|---|---|
| culture of Lactobacillus casei var. rhamnosus | 1800.0 | l |
| potato starch | 6.6 | kg |
| lactose | 3.3 | kg |
| maltose dextrin | 6.0 | kg |

suspension of calcium hydroxide, qs for neutralization to pH = 7.0

Preparation Method:

The culture of Lactobacillus casei var. rhamnosus is centrifuged continuously in a centrifuge to completely eliminate the supernatant. The pellet is recovered under a laminar flow hood and mixed with rapid stirring with the freeze-dried excipients. Next, neutralize the mixture to a pH of 7.0±0.1 using calcium hydroxide.

Freeze Drying

The preparations of cultures of Lactobacillus casei var. rhamnosus for freeze drying are aseptically distributed into stainless steel plates.

It is rapidly frozen to −40° C. then sublimation is carried out at −22° C. When the product has passed the 0° C. threshold, it is oven heated to +37° C.

Depending on the preparations used, the quantities obtained of dry freeze-dried product are approximately as follows:

| | Freeze-dried culture of |
|---|---|
| | Lactobacillus casei var. rhamnosus |
| Simplified medium | 57 kg |
| Enriched medium | 17 kg |

Grinding, Mixing, Packaging

a) Grinding

The freeze-dried Lactobacillus casei var. rhamnosus culture is recovered, ground and sieved through an oscillating type calibration machine provided with a 60 MESH grid in a room with a dehumidified atmosphere (RH less than 50% and filtered (99.999 filtration)); a sample is taken to count the lactobacillae.

The product is stored in hermetically sealed containers.

After titrating the live Lactobacillus casei var. rhamnosus, the products are homogenized to obtain a starting material with the desired titer.

b) Mixing

To obtain batches of freeze-dried culture of Lactobacillus casei var. rhamnosus of 200 to 225 kg with a titer in the range 1×10⁹ to 1×10¹⁰ cells per gram, mixing is carried out in a slow mixer using the following method:
qs of freeze-dried culture of Lactobacillus casei var. rhamnosus on simplified medium;
qs of freeze-dried culture of Lactobacillus casei var. rhamnosus on enriched medium so that the weight of the final mixture is in the range 200 to 225 kg of a powder the titer of which is in the range 1×10⁹ to 1×10¹⁰ cells per gram.

c) Packaging

After mixing, samples are taken as controls. The remaining mixture is packaged as required into suitable hermetically sealed containers.

The powder is then turned into a galenical form.

### EXAMPLE 2

### Study of the Effect on Atopic Dermatitis of a Product Based on Lcr35

The aims of this study are firstly to study the efficacy of Lactobacillus casei var. rhamnosus in association with a prebiotic in the treatment of moderate to severe childhood atopic dermatitis and to evaluate the side effects and clinical tolerance of this treatment.

Type of Study:

Controlled prospective double blind versus placebo study.

Phase 2 study.

Current knowledge.

1) Atopic Dermatitis

Atopic dermatitis is common skin complaint (prevalence estimated between 10% and 25% in Western countries) (2, 3). Its evolution is chronic, frequently with improvement in adolescence. Because of the chronic nature of the prurigenic lesions which are unsightly and necessitate daily care, the moderate and sever forms can have a dramatic psychological effect which may alter parent/child relationships and affect the quality of life of the child and family.

The basic treatment resides in the regular application of emollients associated with a hygieno-dietetic regime. Severe episodes necessitate the application of corticoids or immunosuppressors in a topical form.

The pathogenesis of atopic dermatitis resides in a dysregulation of cellular immunity with an imbalance in the Th1/Th2 ratio, to the advantage of a type Th2 response. There is currently no treatment on the market which can correct this immune dysregulation.

2) Probiotics

Probiotics are microbes in the digestive tract which may have anti allergic properties which encourage a type Th1 response (8, 9). They have been used for nearly 40 years in the symptomatic treatment of diarrhea in children and adults. However, not all probiotics have the same properties. In a study on murine dendritic cells, the bacterium Lactobacillus casei was shown to be a probiotic inducing, at low concentrations, the production of interleukins encouraging the Th1 response (IL-12, IL-6, TNFα) without increasing the production of IL-10 (principal interleukin in the Th2 response) (10).

Probiotics have already been shown to be effective in the primary prevention of atopic disease (11) and to a lesser degree in the treatment of babies (13). These studies were carried out with very young children and the efficacy of the treatment could be due to an initial colonization of intestinal microflora. However, the immunomodulating effects of probiotics are also observed in larger children and adults: they could thus also be effective in these age groups (16). Finally, such treatments prove to be particularly safe as only a few rare cases of endocarditis and septicemia have been described in severely immunosuppressed subjects.

The use of the Lcr35 strain as the source of L. casei offers multiple advantages:
This strain has been sold since 1967 (under the trade name Antibiophilus® then Bacilor®, Jan. 12, 1959, AMM29/03/1974) in the symptomatic treatment of diarrhea.
It has excellent tolerance. No undesirable effects have been observed in France or abroad.
It contains Lactobacillus casei var. rhamnosus (also known as Lactobacillus rhamnosus) which appears now to be a promising probiotic in the treatment and prevention of atopic manifestations.

Materials and Methods

1) Inclusion Criteria

The study was carried out over a period of 17 months. The patients, children aged 2 to 12 years presenting moderate to severe atopic dermatitis, were included in this study after obtaining written consent from their parents and also from children at the age of understanding. The diagnosis of atopic dermatitis was made using standardized criteria (18). Only patients with a SCORAD above 15 were included in the study.

2) Non Inclusion Criteria

Patients with known immunosuppression (congenital or acquired) and those who took or had undertaken treatment which could induce general immunosuppression (systemic corticoids or immunosuppressive drugs) in the preceding three months were not included in the study.

3) Double Blind Study Conditions

The children were randomly distributed into 2 groups.

The first received the symbiotic (containing Lactobacillus casei var. rhamnosus in the form of Bacilor®; the second received only prebiotics (no Lactobacillus casei var. rhamnosus).

Randomization was carried out before the study was begun, depending on the severity of the atopic dermatitis (SCORAD< or >40) and depending on the season (winter, spring, summer, fall).

Sachets containing the probiotics (+prebiotics+metabolites) could be distinguished neither by the patient nor by the physician from sachets containing only prebiotic.

4) Administration Procedure

The symbiotic comprising Lactobacillus casei var. rhamnosus was administered orally in the form of 1.5 g sachets of Bacilor® (produced by Lyocentre Laboratories, Aurillac, France). Each 1.5 g dose was stored in aluminum sachets sealed from air. The doses of symbiotic contained about 1.2×10⁹ colony forming units (or cells) of Lactobacillus rhamnosus Lcr35+a preparation of a specific prebiotic and metabolites secreted by the bacterium.

The prebiotic preparation is derived from the Lactobacillus rhamnosus Lcr35 fermentation medium and contains powdered skimmed milk (on average 0.344 g—bovine protein, 33%, lactose 52%), potato starch (on average 0.759 g) and lactose (on average 0.397 g).

Doses of symbiotic and prebiotic preparations were given in the form of a powder for a drinkable suspension, three times daily for 3 months. The patients were requested to dissolve the preparation in water or in any other preferred cold liquid. None of the patients changed their dietary regime during the period of the study.

During the study period, the patients' normal treatments for atopic dermatitis remained unchanged; all of the patients applied emollients and almost all used dermocorticoids or local immunosuppressors. The topical treatments were noted, as well as all of the other drug intakes whether regular or occasional. The local dermocorticoid and immunosuppressor consumption was evaluated quantitatively by estimating the number of tubes used on each visit.

5) Criteria for Evaluating Treatment Efficacy

Neither the patient nor the parents nor the investigating dermatologist knew the treatment administered to the patient.

Clinical evaluations were performed before the introduction of the treatment, at one month (M1), two months (M2) and three months (M3). Each patient was examined by the same dermatologist on each visit.

The evolution of the symptomology and treatment efficacy were quantified by a global gravity score: SCORAD (SCORing Atopic Dermatitis) developed by the European Task Force for Atopic Dermatitis (19). This strictly clinical score constitutes the reference in evaluating atopic dermatitis treatments.

The SCORAD score combines a clinical evaluation of the intensity and extent of symptoms with a subjective score linked to pruritis and insomnia indicated by the patient, parents or both on a visual scale. The objective SCORAD ranges from 0 to 83. When adding in pruritis and insomnia, the score may then be between 0 and 103.

At the initial visit, the patients were classified into a moderate group (SCORAD between 16 and 39) or severe group (SCORAD more than 40). At each visit, the objective SCORAD and total SCORAD were calculated. The IGA (investigator's global assessment, on a scale of 6 levels including the following categories: 0, nothing; 1, almost nothing; 2, light; 3, moderate; 4, severe; 5, very severe) was also evaluated on each visit. The number of flares and use of local dermocorticoids or immunosuppressors during the preceding month were also noted, as well as possible side effects. During the last visit, the opinion of the patients and/or parents on the treatment efficacy (aggravation, unchanged, improvement) and the tolerance were noted.

Finally, the parents were contacted 6-12 months after the end of the study to determine whether they had observed a resurgence of disease after stopping the treatment.

6) Monitoring Side Effects

Monitoring was exclusively clinical, and monthly.

7) Statistical Analysis

The number of patients necessary to demonstrate a difference of 15 points in the SCORAD between the two groups with a standard deviation of 16, type I error (alpha) of 5%, a power of 80% and a maximum percentage of patients excluded during the clinical test of 20%, was thus 22 persons per group.

The potential differences linked to sex, other atopic diseases and to the use of topical treatments between the two groups was analyzed using a χ² test (or the Fisher's exact test, depending on the case). The baseline (the starting level before any intervention) for age, the number of flares per month, the use of topical treatments, the SCORAD (total) and the objective SCORAD were compared for the two groups using t-test (or the Kruskal-Wallis test for non-parametric data).

At the end of the study period, changes in the SCORAD and objective SCORAD, referred to the baseline in each treatment group, were analyzed using a paired Student t test: the objective SCORAD and the SCORAD between the two groups at M3 were compared using at test for the independent samples. This analysis was also carried out in the sub group of patients presenting other atopic manifestations. Only those finishing the study (primary result and SCORAD available at M3) were included in these analyses.

However, the analyses were repeated over the whole of the population of the study, after having replaced the SCORADs missing at M3 by the mean SCORAD in each treatment group on the basis of data available at M3.

The results for patients in each group who obtained a SCORAD reduction of 50% or more or 90% or more with respect to the baseline at M3 were also analyzed using an exact Fisher test. The IGA scores were analyzed in a similar manner using Wilcoxon and Mann-Whitney tests.

Finally, the total number of flares and the quantities of topical treatments used during the study period were also compared between the two groups for the month preceding the study and for the last month of the study (Kruskal-Wallis test and Friedman test for paired samples).

The statistical analyses were carried out using SPPS for Windows software.

Results:

Population Study:

Of the 48 patients included in the study, nine (symbiotic group, n=7; prebiotic group, n=2; P=0.13) were excluded from the study. The reasons for these exclusions were non presentation of patients for scheduled consultations (symbiotic group, n=5; prebiotic group, n=2) or withdrawal of informed consent (symbiotic group, n=2). Thirty-nine patients (mean age 5.82 years, mean SCORAD=39.7) completed this study. The characteristics of the study population are shown in Table 1. No statistical difference was observed between the two groups on the basis of age, the presence of other atopic manifestations, the monthly number of flares, the use of local dermocorticoids or immunosuppressors and the objective SCORAD and total SCORAD. The base level for the objective SCORAD was 29.1 (symbiotic group=28.7 as opposed to prebiotic group=29.4) and the mean SCORAD reference was 39.2 (symbiotic group=39.1 as opposed to prebiotic group=39.3).

Clinical Effects

The evolution of the objective and overall SCORAD in the two groups is respectively indicated in Tables 2 and 3. In the symbiotic group, the mean SCORAD was 39.1 before treatment (n=24) as opposed to 20.7 after three months of treatment (n=17; P<0.0001, on the basis of 17 complete observations). In the prebiotic group, the mean SCORAD was 39.3 before treatment (n=24) as opposed to 24.0 after three months (n=22; P<0.0001)

The difference between the two treatment groups was clear but statistically small both from the point of view of the objective SCORAD and from the point of view of the SCORAD after 3 months treatment (P=0.418 and P=0.535 respectively). These results are compatible with those obtained in analyses carried out by replacing the missing values for the SCORAD and objective SCORAD at M3.

Since the response to treatment was more pronounced in allergic patients compared with non allergic patients in the first study carried out in children over a year old (17), the inventors also analyzed the results by taking into account the presence or otherwise of other atopic manifestations. Twenty-five patients presented other atopic manifestations (13 in the prebiotic group and 12 in the symbiotic group). At the base level (baseline), the SCORAD was 41.7±11.4 in the prebiotic group as opposed to 49.6±16.8 in the symbiotic group (P=0.181). The two groups showed a substantial decrease in their SCORAD scores after three months treatment: the observed difference between the two groups was not statistically significant, however.

The change in IGA scores was similar to that in the SCORAD scores. In the group receiving symbiotics, the mean IGA score was 3.33 (confidence interval: 2.97-3.70) before treatment (n=24) as opposed to 2.41 (CI: 1.75-3.07) after three months treatment (n=17; P=0.002 on the basis of 17 complete observations).

In the group receiving prebiotics, the mean IGA score was 3.42 (confidence interval 3.20-3.63) before treatment (n=24) as opposed to 2.73 (CI: 2.31-3.14) after three months treatment (n=22; P=0.006).

Use of Topical Drugs

The topical treatments used by the patients were classified into three groups: dermocorticoids (n=34); ointments having a calcineurin inhibitor as the active principle (n=11) and emollients (n=3). Tacrolimus 0.03% ointment was the only product used in the calcineurin inhibitor group. By comparing the consumption of tubes of ointment during the whole period of the study in each of the two groups (prebiotics and symbiotics), no statistical difference was observed (P=0.966). Similar results were obtained by comparing use during the last month of the study (P=0.919). In the two groups, the use of dermocorticoids or pomades with a calcineurin inhibitor (tacrolimus) as the active principle during the preceding month of the study was higher than during month M3 (prebiotic, P=0.005; symbiotic, P=0.07).

Tolerance to Treatment

The treatment was well tolerated in the two groups. No serious side effects were observed; only three incidents of mild abdominal pain were reported—two in the group receiving symbiotic and one in the group receiving prebiotic.

TABLE 1
Characteristics of study population (total and in each group)

| | Total | | Symbiotic | | Prebiotic | | p value |
|---|---|---|---|---|---|---|---|
| Number of patients | 48 | (100.0) | 24 | (100.0) | 24 | (100.0) | |
| Age [yrs, mean (range)] | 5.85 | (2-12) | 5.38 | (2-11) | 6.33 | (2-12) | NS |
| Other atopic diseases | 25 | (52) | 12 | (50) | 13 | (54) | NS |
| Asthma | 17 | (68) | 9 | (75) | 8 | (62) | NS |
| Allergic rhinitis | 19 | (76) | 8 | (67) | 11 | (85) | NS |
| Allergic | 11 | (44) | 5 | (42) | 6 | (46) | NS |
| conjunctivitis | | | | | | | |
| Flares [-month; mean | 7.5 | (0-30) | 8.83 | (0-30) | 6.13 | (0-30) | NS |
| (range)] | | | | | | | |
| Treatments | | | | | | | |
| Dermocorticoids | 34 | (71) | 16 | (67) | 18 | (75) | |
| Class 2 | 29 | (60) | 15 | (63) | 14 | (58) | |
| Class 3 | 5 | (11) | 1 | (4) | 4 | (17) | |
| Tacrolimus, 0.003% | 11 | (23) | 6 | (25) | 5 | (21) | |
| Emollients alone | 3 | (6) | 2 | (8) | 1 | (4) | |
| Monthly use [g; mean | 1.29 | (0-5) | 1.25 | (0-4) | 1.32 | (0-5) | NS |
| (range)] | | | | | | | |
| Base objective SCORAD | 29.0 | (19.9-37.4) | 28.7 | (16.1-36.6) | 29.0 | (21.6-37.7) | NS |
| [median (IQR)] | | | | | | | |
| SCORAD above baseline | 39.7 | (27.6-46.2) | 39.7 | (26.5-46.1) | 39.2 | (30.6-48.1) | NS |
| [median (IQR)] | | | | | | | |

The values given are n (%) unless otherwise indicated.
SCORAD = scoring atopic dermatitis; IQR = interquartile range.

TABLE 2
Evolution in objective SCORAD

| Objective SCORAD | Baseline (n = 24) | 1 month (n = 20) | 2 months (n = 17) | 3 months (n = 17) |
|---|---|---|---|---|
| Symbiotic group | | | | |
| Mean (95% CI) | 29 (22.9-34.5) | 26 (20.2-32.1) | 19 (13.0-25.4) | 16 (8.9-23.8) |
| Median (IQR) | 29 (16.1-36.6) | 28 (15.7-37.2) | 22 (7.5-25.7) | 8 (7.2-32.2) |
| | n = 24 | n = 23 | n = 22 | n = 22 |
| Prebiotic group | | | | |
| Mean (95% CI) | 29 (25.3-33.6) | 25 (18.5-31.1) | 21 (16.0-25.2) | 20 (14.2-25.7) |
| Median (IQR) | 29 (21.6-37.7) | 19 (14.5-38.0) | 19 (13.5-27.4) | 17 (10.1-30.2) |

SCORAD = SCORing Atopic Dermatitis; IQR = interquartile range; CI = confidence interval.

TABLE 3
Evolution in SCORAD

| SCORAD | Base level (n = 24) | 1 month (n = 20) | 2 months (n = 17) | 3 months (n = 17) |
|---|---|---|---|---|
| Symbiotic group | | | | |
| Mean (95% CI) | 39 (31.9-46.4) | 35 (28.5-42.5) | 26 (18.1-34.0) | 21 (11.7-29.8) |
| Median (IQR) | 40 (26.5-46.1) | 37 (23.2-48.6) | 30 (11.5-35.5) | 14 (8.0-39.5) |
| | n = 24 | n = 23 | n = 22 | n = 22 |
| Prebiotic group | | | | |
| Mean (95% CI) | 39 (34.7-43.9) | 32 (24.6-39.2) | 26 (20.8-31.6) | 24 (17.3-30.7) |
| Median (IQR) | 39 (30.6-48.1) | 26 (19.0-46.5) | 26 (18.4-32.5) | 21 (13.4-36.4) |

SCORAD = SCORing Atopic Dermatitis; IQR = interquartile range; CI = confidence interval.

Follow-Up:

All of the children in whom an improvement in atopic dermatitis was observed during treatment, apart from two, underwent a resurgence of disease or at least a deterioration in the condition of the skin in the 2 to 4 months following the end of treatment (either by symbiotics or by prebiotics).

Discussion:

An improvement in atopic dermatitis at the end of the study was observed in both groups of patients (group receiving a prebiotic and group receiving a symbiotic) at a satisfactorily high level. The improvement in the group of patients receiving the symbiotic was statistically only slightly superior to the group of patients receiving the prebiotic.

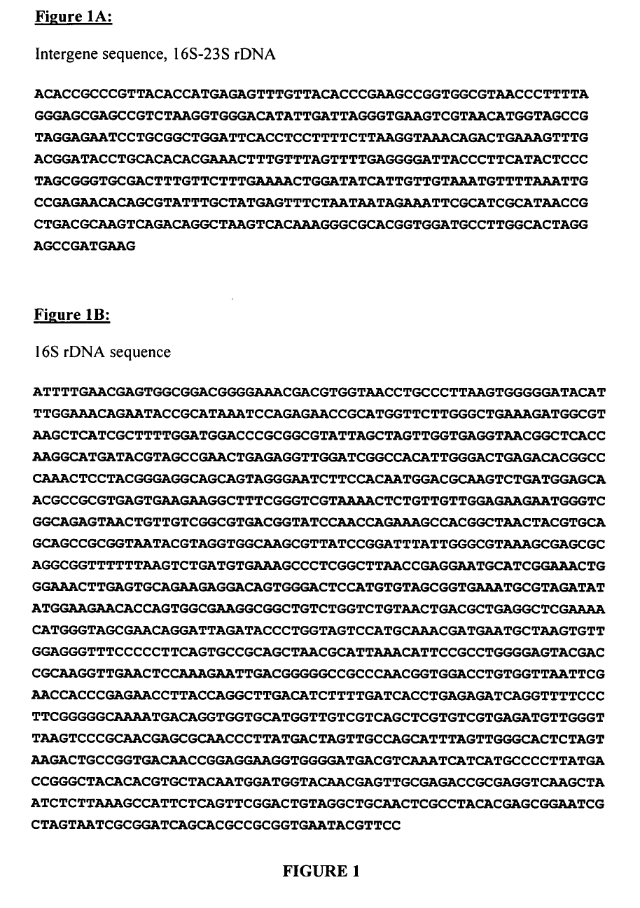

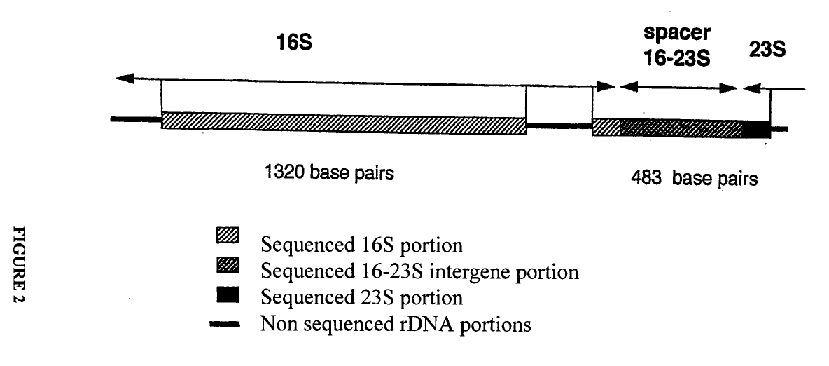

Each probiotic bacterial strain has specific properties and a capacity to proliferate and survive in the digestive tract. The Lactobacillus rhamnosus Lcr35 strain used in the present study has a number of advantageous properties. It has 96% homology with Lactobacillus rhamnosus GG by comparison with 16S-23S sequences. This signature sequence for the Lactobacillus rhamnosus Lcr35 strain is given in FIG. 1.

Further, the strain Lactobacillus rhamnosus Lcr35 has the capacity to ferment lactose, while the strain Lactobacillus rhamnosus GG does not. Finally, previous studies have demonstrated the capacity of Lactobacillus rhamnosus Lcr35 to carry out long term colonization of the intestinal mucosae after its oral administration (20).

The evaluation of the efficacy of symbiotics in the treatment of atopic dermatitis is important as the association with suitable prebiotics can improve the survival rate of probiotic strains following ingestion (21).

Thus, the inventors hypothesized that a prebiotics-probiotics combination could produce better results in the treatment of atopic dermatitis compared with the probiotics alone. Recent results show that lactose and potato starch may be considered to be prebiotics (22, 23). However, the use of the specific prebiotic preparation (as described above) relies on in its ability to enhance the proliferation and survival of the Lactobacillus rhamnosus Lcr35 strain in vitro.

Interestingly, since the prebiotic preparation used is the fermentation medium for Lactobacillus rhamnosus Lcr35 (breeding ground), the symbiotic treatment is composed of the culture medium containing the prebiotics, probiotic and also metabolites secreted by that probiotic. These metabolites may be defined as eubiotics. The combination (probiotic, prebiotic, eubiotic) is present in Bacilor® sold by Lyocentre Laboratories. Its efficacy in the symptomatic treatment of diarrhea is recognized and this product is sold for that indication.

The study period was fixed at 3 months to benefit from a sufficient period to determine the effects of treatment while limiting the probability of interference with a spontaneous variation in the intensity of disease.

The treatment of atopic dermatitis in children aged two years or more by symbiotics [prebiotics+strain of Lactobacillus rhamnosus Lcr35 (1.2×10⁹ colony forming units, three times a day)] was more effective than the treatment composed solely of prebiotics, although the difference was only slight. This is demonstrated both by the change in the SCORAD during the study but also by the subjective evaluation by patients concerning the efficacy of the treatment. Although the severity and importance of lesions remains constant, the treatment with symbiotics appears to have reduced the number of flares and the use of topical drugs. The statistical difference for these two parameters remained small, however.

In fact, a larger reduction in the mean SCORAD was observed in the symbiotic group (20.5 as opposed to 15.6), but this difference was statistically within the significance limits.

Currently, no studies have evaluated the efficacy of symbiotics and prebiotics in the treatment of atopic dermatitis or in any other atopic disease. However, it has recently been demonstrated in patients in good health that prebiotics and probiotics (versus placebos) could significantly reduce the generation and accumulation of potentially toxic fermentation products (24).

Thus, prebiotics themselves may have a major impact on the intestinal flora and may also induce immunological changes. Although supplemental studies are necessary, preliminary data suggests that the consumption of prebiotics may have an influence on parameters linked to cellular immunity and thus modify the Th1/Th2 ratio (25, 26). Hence, specific prebiotics may act on intestinal flora to induce immunomodulation which may be beneficial to the reduction in atopic dermatitis manifestations.

Although the present results demonstrate the importance of prebiotics (especially in combination with probiotics to form symbiotics), factors other than the treatments have been investigated to explain the improvement in the patients' condition.

A seasonal effect can be eliminated as patient inclusion occurred throughout the year, and randomization was carried out to take the seasons into account. However, the frequency of visits and inclusion in a clinical test with the hope of seeing an improvement in disease could have played a favorable psychological role for patients and for their family.

The absence of a comparison with a real placebo (prebiotics cannot be considered to be a placebo) did not allow a definitive conclusion to be drawn; however, regardless of the psychological impact of a placebo, it could not in general induce an improvement in the SCORAD of more than 5 points (13, 17, 27-29). However, in the present study, treatments with the prebiotic and with the symbiotic (and metabolites) both produced a reduction in the SCORAD of more than 15 points (15.6 and 20.5 points respectively).

Further, this improvement could not be linked to an increase in the consumption of dermocorticoids or local immunosuppressors. In contrast, the use of such substances was substantially reduced at the end of the present study. Finally, a resurgence in the disease observed 2 to 4 months following stopping the treatment for almost all patients also suggested that the improvement was not random or to a spontaneous change in atopic dermatitis. All these data confirm the efficacy of prebiotics, especially symbiotics, in the treatment of atopic dermatitis (AD).

The present results show the same trend as the clinical improvement observed in other studies which evaluated the use of probiotics (but not symbiotics) in the treatment of AD. While a large effect was observed in the first of the studies evaluating the efficacy of probiotics in children below two years of age (13), the improvement observed in the second study in which the children were at least one year of age were substantially less significant (17).

The difference in efficacy between these studies (13 and 17) could be explained by the inclusion criteria (“highly specific”) for the patients in the study (13) which were:
patients aged below two years;
with a proven cow's milk allergy.

The observed improvement in the SCORAD in the present study with both prebiotics and symbiotics appears to be greater than that observed in the study carried out by Rosenfeldt (17). This difference could be explained by the difference in the treatment period [6 weeks in Rosenfeldt's study (17) as opposed to 3 months in the present study]. However, it is highly probable that the use of a probiotics/prebiotics/metabolites association also plays an important role in the observed difference. Rosenfeldt's study was carried out with only a probiotic.

However, it should be noted that very little data are available concerning the respective roles of probiotics, prebiotics, symbiotics and metabolites in modulating the immune system. A recent study suggested that probiotics and prebiotics could act via different mechanisms (30).

Two double blind studies with a placebo were recently reported, both being carried out with probiotics to treat children with AD aged below 28 months.

In the first, Lactobacillus GG, a mixture of four strains of probiotics, and a placebo were given to patients with AD suspected of being allergic to cow's milk for 4 weeks (14). No difference was observed between the treated groups either immediately or after 4 weeks of treatment, but in a sub-group [children sensitive to immunoglobulin E (IgE)], the Lactobacillus group showed a statistically superior reduction in the SCORAD with respect to the placebo group. In the second study, L. fermentum and a placebo were administered for 8 weeks (15). The reduction in the SCORAD during the period was significant in the probiotic group but not in the placebo group.

It should be noted that in these two studies, a reduction in SCORAD for the placebo group was observed (−20 and −10 points depending on the study). More precise observation of the placebos used could provide an explanation of the unusual effect of the placebo in the treatment of AD. Cellulose and maltodextrin were respectively used in the studies by Weston et al (15) and Viljanen et al (14). The substances chosen as the placebo were both substances which could be considered to be prebiotics. Thus, the results obtained with such “placebos” in those studies could be similar to those obtained in the prebiotic group in the present study. These results also highlight the importance of choosing a true placebo (and not a compound with prebiotic properties) during clinical tests on AD with prebiotics, probiotics or symbiotics.

In conclusion, a symbiotic compound (Lactobacillus rhamnosus Lcr35 plus a prebiotic) is capable of improving manifestations of atopic dermatitis in children aged two years or more. The amount of improvement observed in this study underlines the importance of prebiotics and symbiotics in the treatment of atopic dermatitis.

### EXAMPLE 3

### Signature Sequence of Lactobacillus rhamnosus Lcr35

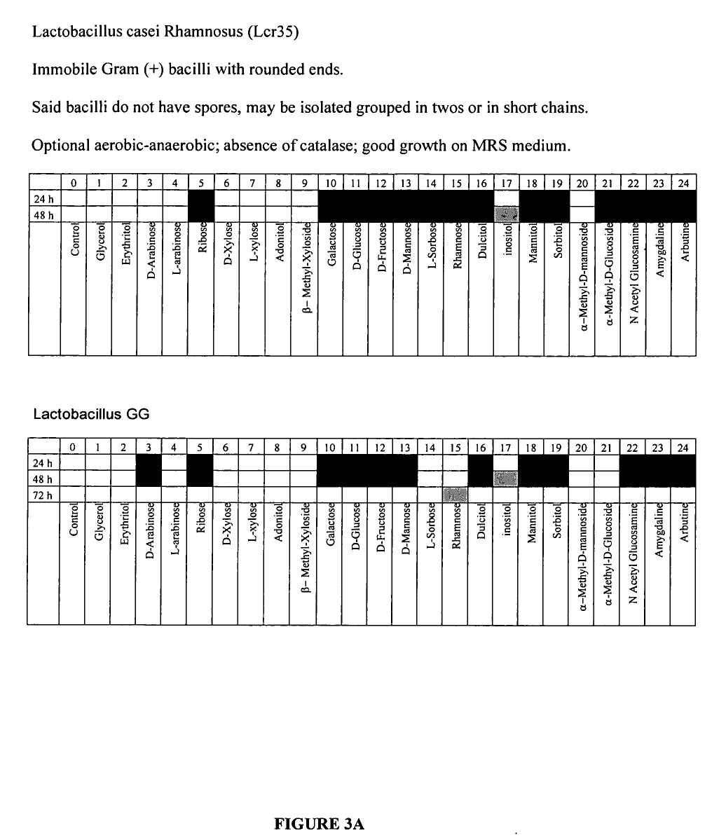

Identification was carried out by PCR then sequencing the gene of 16S ribosomal RNA and the 16-23S intergene portion. The sequencing was double strand sequencing (LI-COR method) followed by alignment with the sequences of other strains. The results and the strategy are shown in FIGS. 1 and 2.

Results of Analysis of 16S Portion Sequence

Position: 16S

Length: 1320 base pairs

Reading ambiguity<5%

| | Identity (%) |
|---|---|
| Lactobacillus casei/rhamnosus | 96 |
| Lactobacillus zeae | 96 |

Results of Analysis of 16-23S Portion Sequence

Position: 16-23S

Length: 483 base pairs

Reading ambiguity<5%

| | Identity (%) |
|---|---|
| Lactobacillus rhamnosus | 96 |
| Lactobacillus casei | 93 |
| Lactobacillus zeae | 93 |

The analyzed strain belongs to the sub-species Lactobacillus casei subsp. rhamnosus. Sequencing of almost the whole of the 16S ribosomal gene could not differentiate Lactobacillus casei subsp casei from Lactobacillus casei subsp. rhamnosus, also termed Lactobacillus rhamnosus.

The signature sequence of Lactobacillus casei subsp. rhamnosus is in the 16-23S intergene portion.

### EXAMPLE 4

### Comparison Between API Profile of Lactobacillus rhamnosus Lcr35 and that of Lactobacillus GG

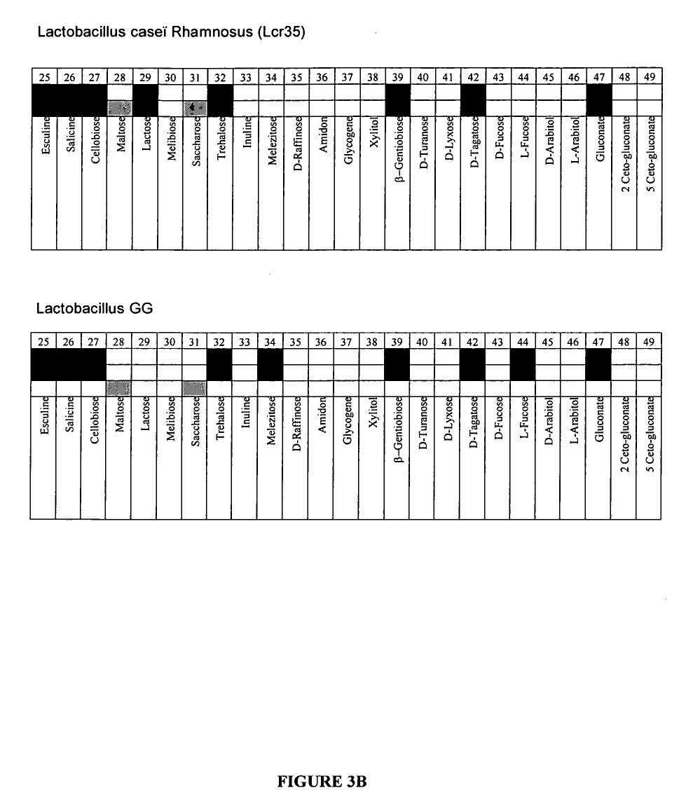

FIGS. 3A and 3B show the API 50 CH metabolic profile of Lactobacillus rhamnosus Lcr35 and Lactobacillus GG strains for times of 24 and 48 hours.

The API 50 CH profile is an analytical strip for studying the metabolism of 49 sugars and is adapted to:
identifying Bacillus with the API 50 CHB medium in about 48 hours;
identifying Lactobacillus with the API 50 CHL medium in 48 hours;
identifying and biotyping enterobacteria with API 50 CHE medium in 48 hours;
any other “analytical” application for studying sugar metabolism.

API(R) identification is well known to the skilled person.

It is clear that the two strains Lactobacillus rhamnosus Lcr35 and Lactobacillus GG do not have exactly the same profile in particular for lactose. A difference in in vitro behavior as regards sugar metabolism implies a difference in behavior in vivo.

### REFERENCES

1. Matricardi P M, Bjorksten B, Bonini S, Bousquet J, Djukanovic R, Dreborg S et al. Microbial products in allergy prevention and therapy. Allergy 2003; 58:461-471.
2. Williams H C. Is the prevalence of atopic dermatitis increasing? Clin Exp Dermatol 1992; 17:385-391.
3. Williams H, Robertson C, Stewart A, Ait-Khaled N, Anabwani G, Anderson R et al. Worldwide variations in the prevalence of symptoms of atopic eczema in the International Study of Asthma and Allergies in Childhood. J Allergy Clin Immunol 1999; 103:125-138.
4. Gehring U, Bolte G, Borte M, Bischof W, Fahlbusch B, Wichmann HE et al. Exposure to endotoxin decreases the risk of atopic eczema in infancy: a cohort study. J Allergy Clin Immunol 2001; 108:847-854.
5. Harris J M, Cullinan P, Williams H C, Mills P, Moffat S, White C et al. Environmental associations with eczema in early life. Br J Dermatol2001; 144:795-802.
6. Kilpi T, Kero J, Jokinen J, Syrjanen R, Takala A K, Hovi T et al. Common respiratory infections early in life may reduce the risk of atopic dermatitis. Clin Infect Dis 2002; 34:620-626.
7. Renz H, Herz U. The bidirectional capacity of bacterial antigens to modulate allergy and asthma. Eur Respir J 2002; 19:158-171.
8. Rautava S, Kalliomaki M, Isolauri E. Probiotics during pregnancy and breastfeeding might confer immunomodulatory protection against atopic disease in the infant. J Allergy Clin Immunol 2002; 109:119-121.
9. Kirjavainen P V, Apostolou E, Salminen S J, Isolauri E. New aspects of probiotics—a novel approach in the management of food allergy. Allergy 1999; 54:909-915.
10. Christensen H R, Frokiaer H, Pestka J J. Lactobacilli differentially modulate expression of cytokines and maturation surface markers in murine dendritic cells. J Immunol 2002; 168:171-178.
11. Kalliomaki M, Salminen S, Arvilommi H, Kero P, Koskinen P, Isolauri E. Probiotics in primary prevention of atopic disease: a randomised placebo-controlled trial. Lancet 2001; 357:1076-1079.
12. Kalliomaki M, Salminen S, Poussa T, Arvilommi H, Isolauri E. Probiotics and prevention of atopic disease: 4-year follow-up of a randomised placebo-controlled trial. Lancet 2003; 361:1869-1871.
13. Isolauri E, Arvola T, Sutas V, Moilanen E, Salminen S. Probiotics in the management of atopic eczema. Clin Exp Allergy 2000; 30:1604-1610.
14. Viljanen M, Savilahti E, Haahtela T, Juntunen-Backman K, Korpela R, Poussa T et al. Probiotics in the treatment of atopic eczema/dermatitis syndrome in infants: a double-blind placebo-controlled trial. Allergy 2005; 60:494-500.
15. Weston S, Halbert A R, Richmond P, Prescott S L. Effects of probiotics on atopic dermatitis: a randomised controlled trial. Arch Dis Child 2005. 4
16. Cross M L, Stevenson L M, Gill H S. Anti-allergy properties of fermented foods: an important immunoregulatory mechanism of lactic acid bacteria? Int Immunopharmacol 2001; 1:891-901.
17. Rosenfeldt V, Benfeldt E, Nielsen S D, Michaelsen K F, Jeppesen D L, Valerius N H et al. Effect of probiotic Lactobacillus strains in children with atopic dermatitis. J Allergy Clin Immunol 2003; 111:389-395.
18. Williams H C. On the definition and epidemiology of atopic dermatitis. Dermatol Clin 1995; 13:649-657.
19. Kunz B, Oranje A P, Labreze L, Stalder J F, Ring J, Taieb A. Clinical validation and guidelines for the SCORAD index: consensus report of the European Task Force on Atopic Dermatitis. Dermatology 1997; 195:10-19.
20. de Champs C, Maroncle N, Balestrino D, Rich C, Forestier C. Persistence of colonization of intestinal mucosa by a probiotic strain, Lactobacillus casei subsp. rhamnosus Lcr35, after oral consumption. J Clin Microbiol 2003; 41: 1270-1273.
21. Kailasapathy K, Chin J. Survival and therapeutic potential of probiotic organisms with reference to Lactobacillus acidophilus and Bifidobacterium spp. Immunol Cell Biol 2000; 78:80-88.
22. Szilagyi A. Review article: Lactose—a potential prebiotic. Aliment Pharmacol Ther, 2002; 16:1591-1602.
23. Szilagyi A. Redefining lactose as a conditional prebiotic. Can J Gastroenterol 2004; 18:163-167.
24. De Preter V, Geboes K, Verbrugghe K, De Vuyst L, Vanhoutte T, Huys G et al. The in vivo use of the stable isotope-labelled biomarkers lactose-[15N]ureide and [2H4]tyrosine to assess the effects of pro- and prebiotics on the intestinal flora of healthy human volunteers. Br J Nutr 2004; 92:439-446.
25. Schley P O, Field C J. The immune-enhancing effects of dietary fibres and prebiotics. Br J Nutr 2002; 87(Suppl. 2):S221-230.
26. Boehm G, Jelinek J, Knol J, M′Rabet L, Stahl B, Vos P et al. Prebiotics and immune responses. J Pediatr Gastroenterol Nutr 2004; 39(Suppl. 3):S772-773.
27. Salek M S, Finlay A Y, Luscombe D K, Allen B R, Berth-Jones J, Camp RD et al. Cyclosporin greatly improves the quality of life of adults with severe atopic dermatitis. A randomized, double-blind, placebo-controlled trial. Br J Dermatoll993; 129:422-430.
28. van Joost T, Heule F, Korstanje M, van der Broek M J, Stenveld H J, van Vloten W A. Cyclosporin in atopic dermatitis: a multicentre placebo-controlled study. Br J Dermatol 1994; 130:634-640.
29. Gutgesell C, Heise S, Seubert S, Seubert A, Domhof S, Brunner E et al. Double-blind placebo-controlled house dust mite control measures in adult patients with atopic dermatitis. Br J Dermatol, 2001; 145:70-74.
30. Roller M, Rechkemmer G, Watzl B. Prebiotic insulin-enriched with oligofructose in combination with the probiotics Lactobacillus rhamnosus and Bifidobacterium lactis modulates intestinal immune functions in rats. J Nutr 2004; 134:153-156.
31. Forestier C, De Champs C, Vatoux C, Joly B. Probiotic activities of Lactobacillus casei rhamnosus: in vitro adherence to intestinal cells and antimicrobial properties. Res. Microbiol. 2001; 152: 167-173.

1-15. (canceled)

16. A method of treating a patient with atopic dermatitis which comprises administering to the patient a drug or a food supplement comprising a Lactobacillus rhamnosus bacterium in live form and lactose, wherein said bacterium is capable of fermenting lactose.

17. The method of claim 16, wherein the lactose is in the form of a lactose-based medium.

18. The method of claim 16, wherein said bacterium is Lactobacillus rhamnosus Lcr35.

19. The method of claim 16, wherein the product is freeze-dried.

20. The method of claim 17, wherein the lactose-based medium contains potato starch.

21. The method of claim 17, wherein the bacterium has been cultured on said medium for at least 24 hours.

22. The method of claim 21, wherein the bacterium has been cultured on said medium for at least 48 hours.

23. The method of claim 21, wherein the bacterium has been cultured on said medium for about 72 hours.

24. The method of claim 16, wherein the patient is a human being below 15 years of age.

25. The method of claim 16, wherein the patient is a human being over two years of age.

26. The method of claim 16, wherein the patient has a lactose allergy.

27. The method of claim 21, wherein the product comprises a concentration of bacterium in the medium in the range 1×10⁸ to 1×10¹⁰ colony forming units per ml or per gram of frozen or dry product.

28. The method of claim 16, wherein the drug or food supplement comprises 1×10⁸ cells per gram or more of bacteria.

29. The method of claim 16, wherein the drug or the food supplement comprises in the range 1×10⁸ to 1×10¹⁰ cells per gram.

30. The method of claim 16, wherein the treatment period is in the range of 2 weeks to 1 year, preferably in the range of 2 weeks to 6 months.


SEQUENCE LISTING (2 sequences)

SEQ ID NO: 1 - DNA, 431 bp, Lactobacillus rhamnosus
acaccgcccgttacaccatgagagtttgttacacccgaagccggtggcgtaacccttttagggagcgagccgtctaaggtgggacatattgattagggtgaagtcgtaacatggtagccgtaggagaatcctgcggctggattcacctccttttcttaaggtaaacagactgaaagtttgacggatacctgcacacacgaaactttgtttagttttgaggggattacccttcatactccctagcgggtgcgactttgttctttgaaaactggatatcattgttgtaaatgttttaaattgccgagaacacagcgtatttgctatgagtttctaataatagaaattcgcatcgcataaccgctgacgcaagtcagacaggctaagtcacaaagggcgcacggtggatgccttggcactaggagccgatgaag

SEQ ID NO: 2 - DNA, 1280 bp, Lactobacillus rhamnosus
attttgaacgagtggcggacggggaaacgacgtggtaacctgcccttaagtgggggatacatttggaaacagaataccgcataaatccagagaaccgcatggttcttgggctgaaagatggcgtaagctcatcgcttttggatggacccgcggcgtattagctagttggtgaggtaacggctcaccaaggcatgatacgtagccgaactgagaggttggatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatgggtcggcagagtaactgttgtcggcgtgacggtatccaaccagaaagccacggctaactacgtgcagcagccgcggtaatacgtaggtggcaagcgttatccggatttattgggcgtaaagcgagcgcaggcggttttttaagtctgatgtgaaagccctcggcttaaccgaggaatgcatcggaaactgggaaacttgagtgcagaagaggacagtgggactccatgtgtagcggtgaaatgcgtagatatatggaagaacaccagtggcgaaggcggctgtctggtctgtaactgacgctgaggctcgaaaacatgggtagcgaacaggattagataccctggtagtccatgcaaacgatgaatgctaagtgttggagggtttcccccttcagtgccgcagctaacgcattaaacattccgcctggggagtacgaccgcaaggttgaactccaaagaattgacgggggccgcccaacggtggacctgtggttaattcgaaccacccgagaaccttaccaggcttgacatcttttgatcacctgagagatcaggttttcccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaccggaggaaggtggggatgacgtcaaatcatcatgccccttatgaccgggctacacacgtgctacaatggatggtacaacgagttgcgagaccgcgaggtcaagctaatctcttaaagccattctcagttcggactgtaggctgcaactcgcctacacgagcggaatcgctagtaatcgcggatcagcacgccgcggtgaatacgttcc